(19) United States
(12) Patent Application Publication
(10) Pub. No.: US 2021/0353399 A1
(43) Pub. Date: Nov. 18, 2021

(54) SYSTEM AND METHOD OF USE FOR CAROTID STENTING

(71) Applicant: Shereef Hilmy, MISSION, TX (US)

(72) Inventor: Shereef Hilmy, MISSION, TX (US)

(21) Appl. No.: 17/316,420

(22) Filed: May 10, 2021

Related U.S. Application Data
(60) Provisional application No. 63/023,336, filed on May 12, 2020.

(51) Int. Cl. A61F 2/01; A61B 17/12; A61F 2/966

(52) U.S. Cl. CPC A61F 2/014; A61B 17/12109; A61B 17/12136; A61M 25/0097; A61F 2/966; A61B 17/1204; A61F 2/011

(57) ABSTRACT

The present invention is directed to a medical system and its method of use for percutaneous carotid stenting. The method utilizes flow reversal of the carotid artery and achieves it via a new and improved system that requires a non-invasive procedure. The medical system invention obviates the need for a surgical incision in the neck of a patient when performing conventional carotid surgery. Moreover, the procedure requires no manipulation of the carotid lesion or artery prior to establishing retrograde blood flow. The system presented in this invention improves upon standard carotid artery stenting, which has been plagued by a higher incidence of stroke due to the distal plaque embolization. The carotid stenting system may include at least one catheter at least one wire, a self-expanding stent, at least one arterial sheath, a one venous sheath, at least one control valve, and at least one filtering apparatus.

### CROSS-REFERENCE TO RELATED APPLICATIONS

This application claims the benefit of U.S. Provisional Patent Application Ser. No. 63/023,336, filed on May 12, 2020, which is incorporated herein in its entirety.

### FIELD OF THE INVENTION

The present invention relates generally to medical systems and methods of use, and more particularly, to a medical system and its method of use for percutaneous carotid stenting.

### BACKGROUND OF THE INVENTION

Numerous administrations have tried to solve, or at least mitigate, the health problems plaguing millions of Americans. A number of creative interventions have been implemented in order to treat the illnesses that not only affect Americans, but also hundreds of millions of individuals around the world. In relation to the U.S., most of these complications arise from practicing poor dietary habits in combination with a sedentary lifestyle. This ultimately leads to obesity, diabetes, hypertension and atherosclerosis, which result in quicker deterioration of health. According to a report published in the National Center for Biotechnology Information, it is foreseeable that at some point in the future, half of all adults in the United States will be obese. This is largely the result of the diet being followed, as the U.S. Department of Health and Human Services stated, the typical American diet exceeds the recommended intake levels or limits in four categories: calories from solid fats, added sugars, refined grains, sodium, and saturated fat.

Coupled with the lack of nutritional awareness, Americans lack the motivation or time to practice a healthy, active lifestyle, and instead prescribe to a sedentary one. A report by the U.S. Department of Health and Human Services found that less than 5% of adults participate in 30 minutes of physical activity each day. The consequences of this lifestyle are unavoidable—because it leads to increased risk of cardiovascular diseases, obesity, strokes, diabetes, and many other illnesses. Such sedentary lifestyle coupled with an unhealthy diet exacerbates the problems even further. This has contributed to the development of the metabolic syndrome and the growing “obesity epidemic” in the U.S.

Further, it would be naïve to attribute these consequences to actions of individuals alone, and forget to hold businesses' productivity requirements accountable for this epidemic. Americans are highly productive in comparison to citizens of other countries, meaning their daily routine centers around their career for more hours than most individuals worldwide. This undoubtedly shaves hours off their day that could be utilized to perform physical activities, cook healthier meals, and work on their mental health. Not only do they have less time available, but they are also more exhausted and desire a sedentary usage of their diminished free time upon arriving home from work.

As mentioned previously, the increased risk of developing a disease is what makes a sedentary lifestyle along with an unhealthy diet so dangerous. Of these diseases, those that affect the arterial circulation pose greater danger, as they can lead to failure of supplying the vital organs, particularly the brain with oxygen, leading to a stroke and other complications. One of these potentially fatal diseases that must be treated expeditiously is arterial disease, with a special focus on carotid artery disease (CAD). Carotid artery disease, results from the formation of cholesterol plaque that lodges on the wall of the carotid artery, limiting blood flow to the brain and face. If this persists and the plaque deposit amasses, a major source of blood that supplies the face and brain may cease and the patient may experience symptoms of dizziness, sudden weakness or numbness to the area lacking blood supply, vision problems, among others. Moreover, cholesterol plaque can embolize and lodge in the brain circulation causing a major stroke or TIA (i.e., mini stroke).

In order to properly treat the patient of such disabling and at times life-threatening condition, surgical procedures have been developed but are far from optimal. In one case, the surgeon may perform a carotid endarterectomy, where a surgical incision is made in the neck and the plaque is manually removed to allow for proper blood flow to the brain. Another method includes the percutaneous placement of a stent in the clogged area which expands the artery and allows uninterrupted blood flow. Other methods allow for the insertion of a filter so that any debris that becomes dislodged during the procedure does not travel to the brain and potentially blocks blood flow, leading to a periprocedural stroke.

While these methods have been proven to save the lives of countless patients with CAD, they are inefficient in many aspects. For example, they are all highly invasive surgeries, requiring a surgical incision in the neck and artery, exposing the neck and artery to external contamination, bleeding, infection, nerve injury and all other risks inherent in any invasive surgery. Another way in which they are inefficient is that there is no redundant method of collecting dislodged debris during the surgery.

When taking into account the prevalence of Americans that have heart and vascular disease of some form, or are likely to develop one in the future given their sedentary lifestyle and unhealthy diet, it is of utmost importance to develop a more efficient surgical procedure for carotid artery disease than those available. Accordingly, there is an established need, but as of yet unmet, for the development of a minimally invasive procedure that allows for redundancy in prevention of a stroke.

### SUMMARY OF THE INVENTION

The present is directed to a medical system and its method of use for percutaneous carotid stenting. The medical system achieves flow reversal of the carotid artery via a new and improved method that establishes blood flow reversal through non-invasive procedure.

Introducing a first embodiment of the invention, a carotid stenting system for carotid artery stent placement through trans-femoral access, comprising:

a sheath introducible into a femoral artery at an access site, the sheath providing a general tubular body defining a lumen and having a proximal end, a distal end, a sidewall construction that includes an embedded passageway, and a complaint balloon positioned proximate to the sheath's distal end,
wherein the sheath is of a sufficient length to reach a carotid artery from the access site;

a venous sheath including a connection port at a proximal end, the venous sheath introducible into a femoral vein;

a flexible tube linking the sheath to the venous sheath creating a retrograde blood flow path from the carotid artery to the femoral vein; and

a filtration device intercepting the retrograde blood flow path and configured to capture impurities traversing the retrograde blood flow path,
wherein the sheath allows the advancement of a plurality of devices through the sheath's lumen needed to place a stent in the carotid artery.

In another aspect, the carotid stenting system may include a filter positioned about the distal end of a wire positionable superior to the stent.

In another aspect, the sheath may include a marker about its distal end.

In another aspect, the sheath may include a connection hub having a plurality of ports. The connection hub may also include an inflation port in communication with the passageway of the arterial sheath and connectable to an inflation device, such as a syringe, having an injectable fluid, such as saline. When fluid volume is pushed into the passageway of the arterial sheath, the complaint balloon inflates and occludes the carotid artery. This prevents antegrade flow in the carotid artery.

In another aspect, the sheath may be percutaneously positioned inside of the femoral artery, and the venous sheath may be percutaneously positioned inside of the femoral vein.

In another aspect, the system may include an arterial sheath that is about 12 cm and 6 F sheath. The arterial sheath may be percutaneously cannulated to the femoral artery in retrograde fashion approximately about 2 cm through about 4 cm inferior to a patient's inguinal ligament.

In another aspect, the sheath may be 90 cm long, and the venous sheath in some embodiments may be a 8 F sheath.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a top plan view showing a first embodiment of the percutaneous carotid stenting system of the present invention;

FIG. 2 presents a top plan view of the saline and/or contrast balloon inflation port of the percutaneous carotid stenting system of the present invention;

FIG. 3 presents a sheath inside of a femoral artery advanced superiorly to the common carotid artery and positioned inferior to the lesion and a sheath inside of the femoral vein, illustrating one of the steps of the method of use of the present invention.

FIG. 4 presents a wire and sheath inside of a common carotid artery, illustrating one of the steps of the method of use of the present invention;

FIG. 5 presents an exemplary embodiment of the sheath that includes a balloon port lumen, the sheath is utilized with the percutaneous carotid stenting system of the present invention;

FIG. 6 presents the carotid stenting system of FIG. 1, inside of the common carotid artery being fed into the internal carotid artery;

FIG. 7 presents the carotid stenting system of FIG. 1, deploying a filter above the lesion inside of the carotid artery; and

FIG. 8 presents the carotid stenting system of FIG. 1, applying a stent to the lesion inside of the carotid artery with the filter in the open position.

Like reference numerals refer to like parts throughout the several views of the drawings.

### DETAILED DESCRIPTION

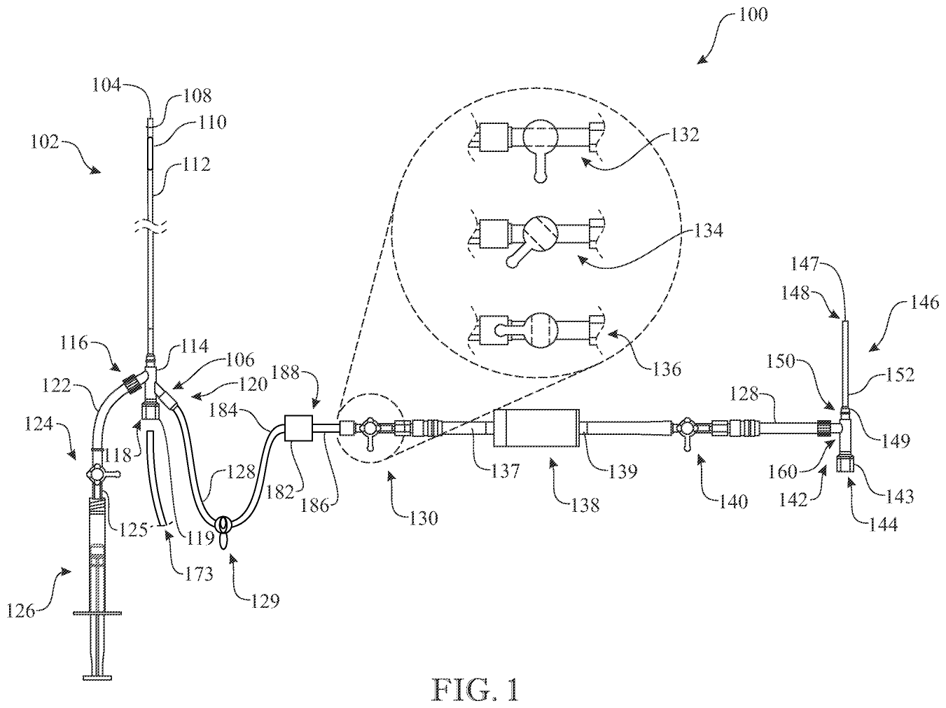

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed to a medical system and its method of use for percutaneous carotid stenting. The method utilizes flow reversal of the carotid artery and achieves it via a new and improved system that requires non-invasive procedure. For instance, this new invention obviates the need to make a surgical incision in the neck of a patient and the need to administer general anesthesia to the patient. Moreover, the procedure requires no manipulation of the carotid lesion or artery prior to establishing blood flow reversal.

Initially, one will appreciate that the following description of the medical system and method of use may be generally used with high risk, symptomatic or asymptomatic patients. High risk patient's may include: a patient who has a cranial nerve injury, has had head or neck surgery, has surgically inaccessible lesion, had prior neck radiation, spinal immobility fusion, bilateral carotid disease requiring alternative treatments, laryngectomy, tracheostomy, hostile neck, is over the age of 75, has suffered from pulmonary disease, cardiomyopathy, unstable angina, abnormal stress tests, congestive heart failure, poorly controlled diabetes, prior carotid endarterectomy with restenosis, and patients requiring major surgery or open heart surgery, including vascular surgery.

In one exemplary embodiment, a patient must go through a series of pre-procedural steps before a physician goes through the procedure of applying a stent to the carotid lesion. For instance, a patient will ideally be started on dual antiplatelet therapy—that stops platelets from sticking together and forming blood clots—and statin therapy—that lowers cholesterol levels in the body and prevents further buildup of plaque—at least one week before the procedure. Alternatively, in medical emergencies, the patient may be given a high dosage of blood thinners approximately 12 hours before the procedure. The blood thinners may include ASA (medication that contains aspirin), Plavix®, or the like. If necessary, a CT angiogram of the carotid arteries or a formal carotid angiogram should be performed if the person has not done one within 3 to 6 months of the procedure.

Referring now to FIGS. 1, 2, and 5, a stenting medical system, generally designated 100, in accordance with aspects of the present invention is shown. It is readily understood by those skilled in the art that the present embodiment of the present invention may be employable for any applicable carotid stenting procedure that does not fall within the limitations set forth above. Furthermore, Applicant has decided to focus, as an example, but not to be limited, to a stent procedure to treat the carotid artery of a patient. Therefore, for clarity the “stenting medical system” is herein after referred to as a “carotid stenting system.” The carotid stenting system generally comprises in its simplest form an arterial sheath 102, an inflation device 126, a three-way stopcock 129, a primary control valve 130, a filtering system 138, a secondary valve 140, and a venous sheath 146, and at least one catheter 173.

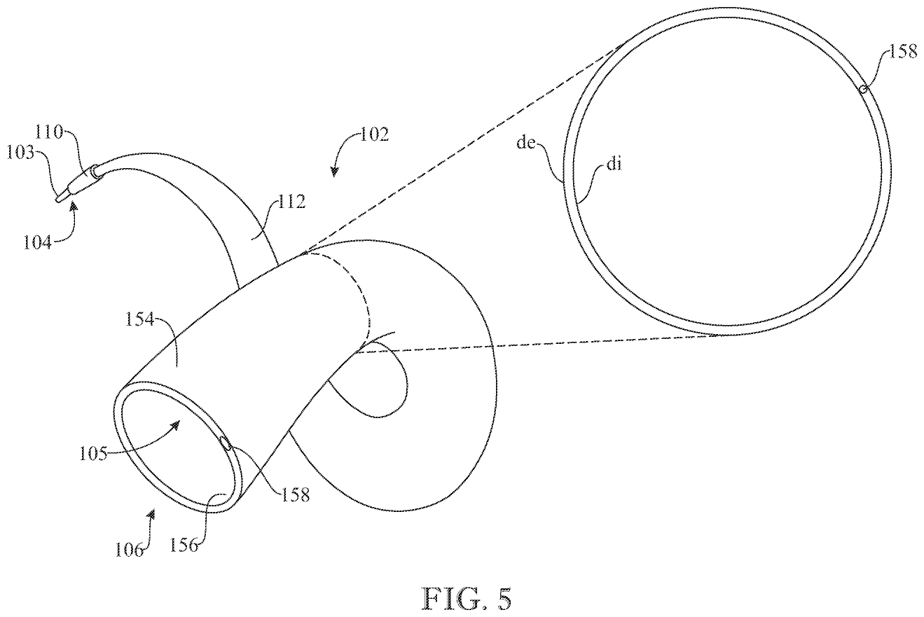

With reference to FIGS. 1 and 5, the arterial sheath 102 of the carotid stenting medical system 100 comprises a multi-layer covering 112 that extends between a distal end 104 and a proximal end 106 and includes two opposite open ends 103, 105. The arterial sheath, in one exemplary form, but not to be limited, may have an approximate length of about 90 cm measured from the distal end 104 of the sheath to its proximal end 106. Although the arterial sheath's size and length may vary depending on the prognosis and needs of the patient, the preferred size of the sheath will be an 8 F-sized sheath. A sheath this size includes an interior surface 156 diameter di of about 2.90 mm and an exterior surface 154 diameter de of about 3.32 mm. In another exemplary form, the arterial sheath 102 may be coated with a hydrophilic coating and include a balloon port lumen 158. Proximate the distal end 104 of the arterial sheath 102 is a marker 108 that is highly visible with imaging equipment, such as a fluoroscope. In one exemplary embodiment, the marker may be made out of a gold coil and be positioned about 2-5 mm proximal to the distal tip of the arterial sheath 102. The arterial sheath may further include an inflatable complaint balloon 110 disposed about the sheath's exterior surface 154. As shown in FIG. 1, the complaint balloon may be positioned a distance of about 5 mm proximal to the distal end 104 of the arterial sheath 102, and may be made out of polyurethane or silicone material. It should be readily understood, however, that alternative materials are employable. The compliant balloon is inflatable by volume, rather than pressure and can fully conform to an artery. In an exemplary embodiment, the complaint soft balloon 110 may expand to about 12-15 mm when inflated. When the soft balloon 110 is deflated, the balloon does not extend beyond the outer side surface of the sheath. Moreover, the arterial sheath, as well as the venous sheath, each include an inner dilator to facilitate its introduction and advancement into the selected vessel in an atraumatic fashion.

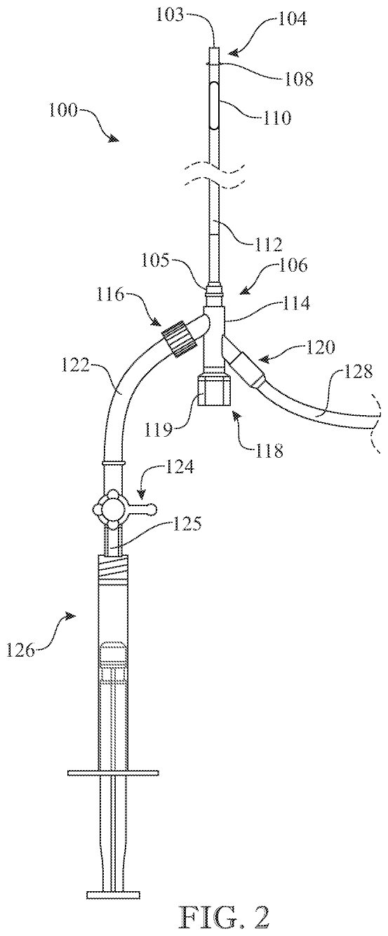

Returning now to FIGS. 1 and 2, the arterial sheath 102 of the medical system 100 connects to a connection hub 114 that generally includes three ports. The first port 116 connects to one end of a connector hose 122, with the opposite end of the connector hose 122 connecting to a clamp or control valve 124. The valve 124 sequentially connects to a port 125 that is connectable to an inflation device 126. In one exemplary form, the port 125 may include a locking mechanism, such as a luer lock, that engages and securely connects to the distal end of the inflation device 126. The inflation device 126, in a preferred exemplary embodiment, but not to be limited to, is a syringe having injectable fluid stored therein, such as a mixture of saline and contrast. The inflation device 126 of the carotid stenting system 100 is used to manipulate the compliant balloon provided at the distal end 104 of the arterial sheath 102. For example, to inflate the balloon 110, a physician may use the input device 126 to inject or otherwise introduce fluid volume into the balloon 110, thereby causing the balloon to expand to a desirable size in order to temporarily occlude the carotid artery to prevent antegrade flow, hence minimizing plaque embolization. Similarly, the physician may use the inflation device 126 to deflate the balloon by using the inflation device 126 to cause a retrograde vacuum that sucks out or otherwise removes the fluid volume inside of the balloon, causing the expanded balloon to condense until the balloon reaches its original size. The clamp or stop-cock valve 124 that connects to the sheath 122 prevents air from entering the patient or system 100 that could otherwise lead to a fatal air embolism.

As aforementioned, the connection hub 114 of the carotid stenting system 100 includes at least a secondary port 118 and a third port 120. The secondary port 118 generally includes a cross-cut valve 119 that allows the insertion of objects, such as a filter wire, a balloon, or a stent through the lumen of the arterial sheath 112 and into the patient's blood vessel. The third port 120 of the connection hub 114 connects to a connection hose 128 on one end and a three way stopcock valve 129 on the other. The stopcock valve 129 may be used an access point to inject contrast into the patient and/or connect another apparatus. For instance, in one exemplary embodiment, a pump system 182 may be attached to the three way stopcock 129 through a connector hose 184 on a first end. Opposite the first end, the second end on the pump 182 is connected to a control valve 130 with the use of a secondary connector hose 186. One will appreciate that the length of connector hoses 128, 184 should be long enough to provide the system with flexibility and increased functionality, particularly when an operator is handling the three way stopcock 129 to inject contrast into the patient. The control valve 130 is used to control the flow rate of fluid flowing through the system. As is best illustrated in FIG. 1, the control valve 130 can be positioned in a fully-open position 132, a partially-open position 134, and a fully-closed position 136.

With Continued reference to FIG. 1, the filtering apparatus 138 of the present invention includes a filtering end 137, and an opposite filtered end 139. The filtering end 137 of the filtering apparatus 138 connects to the main control valve 130 of the carotid stenting system 100. The filtering apparatus 138 is designed or otherwise configured to trap any impurities flowing from the filtering end 137 and out of the filtered end 139 of the apparatus 138. Impurities include, but are not limited to, plaque or lesion deposits that may break off the lesion during the stenting process. The opposing, filtered end 139, of the filtering apparatus 138 connects to a secondary control valve 140. The secondary control valve 140, which generally comprises the venous sheath stopcock serving as the control valve, generally provides three general positions, fully-open in two directions, or fully-closed. It should be readily understood, however, that alternative control valves that include separate but similar features as the ones described herein may be utilized to replace the described primary and secondary control valves of the present system without departing from the scope of the invention. Accordingly, the aforementioned description of the control valves is understood to be exemplary and should not be considered limiting.

Opposite the arterial sheath 102, the carotid stenting system 100 includes a venous sheath 146 comprising a covering or sheath 152 that extends between a distal end 148 and a proximal end 150 and having two opposite open ends. The venous sheath, in one exemplary form, but not to be limited to, may have an approximate length of about 12 cm measured from the distal end 147 of the sheath to its proximal end 149. Although the venous sheath's length and size may vary depending on the prognosis and need of the patient, in one exemplary embodiment, the size of the sheath will be an 8 F-sized that provides an internal diameter of about 2.90 mm and an exterior diameter of about 3.30 mm sheath. A sheath of that size is introduced in the femoral vein of the patient.

As shown in FIG. 1, the venous sheath 146 connects to a connection hub 142 that generally includes at least two ports. The first port 160 connects to the connector hose 128 that connects to the secondary control valve 140. In one exemplary form, the port 160 may include a locking mechanism, or the like, that engages and securely connects to the connector hose 128 thereto. The second port 144 on connection hub 150 generally includes a valve 143 that allows the insertion of objects through the internal passageway provided by the venous sheath 146 and into the patient's blood vessel.

With Reference now to FIGS. 1-8, the method of use of the carotid stenting system 100 is described in one exemplary form.

With particular reference to FIGS. 1, 3, 4, and 6-8, after a patient goes through the pre-procedural steps generally outlined herein above, a physician may begin the method by applying a local anesthetic to an access site, or in this case the femoral access site 200. In one exemplary form, the access site 200 is located approximately 2-4 cm inferior to the patient's inguinal ligament. An anesthetic agent is injected in the area of the femoral vessels to prevent the patient from feeling any pain throughout procedure. After the application of a local anesthetic to the access site, the femoral vein of the patient is percutaneously cannulated through the modified Seldinger technique. The modified Seldinger technique generally includes a needle being introduced into the patient's femoral vein. A wire is then fed through the needle and into the vein before the needle is removed. The venous sheath 146, along with its introducer, are then advanced over the wire and into the patient's vein 206 along the direction of venous blood flow. In one exemplary form, the sheath introduced into the patient's femoral vein is an 8 F sheath. After or before the femoral vein 206 of the patient is cannulated, the femoral artery 208 of the patient is percutaneously cannulated using the modified Seldinger technique. Unlike the venous sheath 146, which was introduced in the direction of blood flow (i.e., toward the heart), an arterial sheath is introduced in the opposite direction of blood flow, i.e., in a retrograde fashion in the femoral artery 208. The arterial sheath, in one exemplary form, is a 12 cm 6 F sheath.

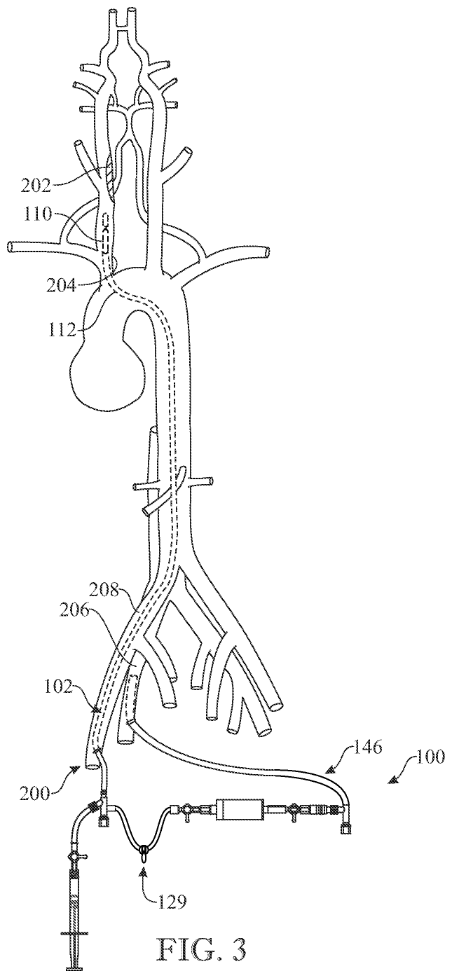

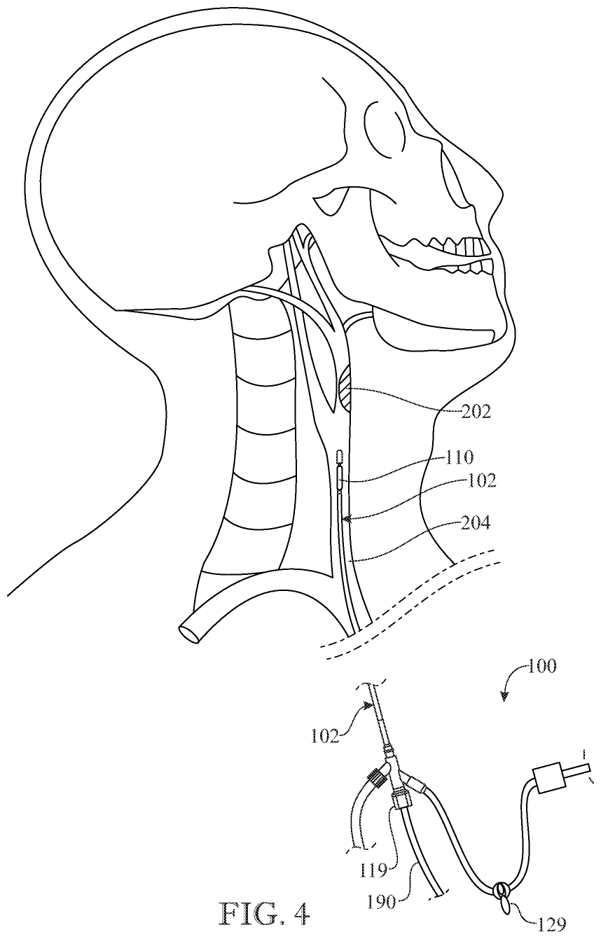

Turning now to FIGS. 3 and 4, a stiff wire and appropriate catheter is introduced through the valve 119 into the lumen of the sheath positioned in the femoral artery 208 and advanced into the arterial circulation. The wire with the accompanying catheter is then advanced to the desired location, which for the purpose of this example is the carotid artery. Accordingly, the wire is advanced through the femoral artery sheath, past the aortic arch, and into the common carotid artery 204 intended to be treated. One will appreciate that the wire may be positioned in either the right or left carotid artery and thus, the description of the method provided herein should be interpreted as exemplary and not limited. After the wire is advanced and the distal end of the wire is at the carotid artery 204, a catheter is fed through the sheath and over the wire, the wire is then removed and contrast is injected to view the placement of the catheter in an angiogram. After the catheter position is confirmed to be located in the carotid artery 204, a an angled stiff wire 190 is introduced. In one exemplary form, the angled stiff wire may be a 0.035 mm wire. The wire 190 is advanced until it reaches the lesion 202 intended for treatment. When placing the wire 190, the main goal is to avoid crossing or passing the lesion otherwise, complications, such as embolization may ensue.

After the stiff wire 190 is in place, the catheter and (6 F) sheath are removed from the patient while applying manual pressure at the access site 200 in the femoral artery to prevent bleeding, and a femoral catheter or sheath 102 is introduced and advanced over the stiff wire 190. The femoral sheath 102, in one exemplary embodiment, is a 90 cm, 8 F sheath that is fed into the femoral artery through the aortic arch and into the common carotid artery. The stiff wire 190 is then removed from the patient. In one exemplary form, the femoral sheath 102 may be coated with a hydrophilic coating and may include a balloon port lumen 158 and a marker 108 that is visible to imaging equipment, such as a fluoroscope (See, FIGS. 1 and 5). Again, contrast may be utilized to confirm the position of the sheath 102 with respect to the lesion 202. The patient undergoing the procedure, should be heparinized to an activated coagulation time (ACT) within a therapeutic range of about 250-300 seconds prior to the insertion of the stent.

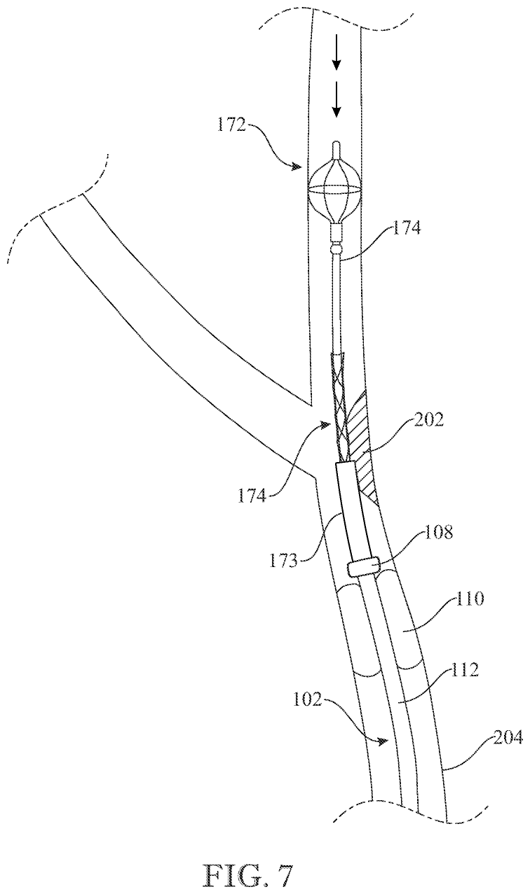

Referring now to FIGS. 1, 3 and 6-9, after the femoral sheath 102 is advanced in the femoral artery 208 and positioned proximal to the lesion 202 in the common carotid artery 204 of the patient, and the venous sheath 146 is positioned in the femoral vein 206, the complaint balloon 110 is inflated to occlude the carotid artery 208. A wire 172 that may include a filter 172 (i.e., filter wire) is advanced through the femoral sheath 102 and positioned superior to the lesion 202 before the filter is deployed (see FIG. 7). This step, however, should be performed after flow reversal has been initiated. One will appreciate that the filter 172 is used to capture any impurities that may flow toward the brain during the procedure.

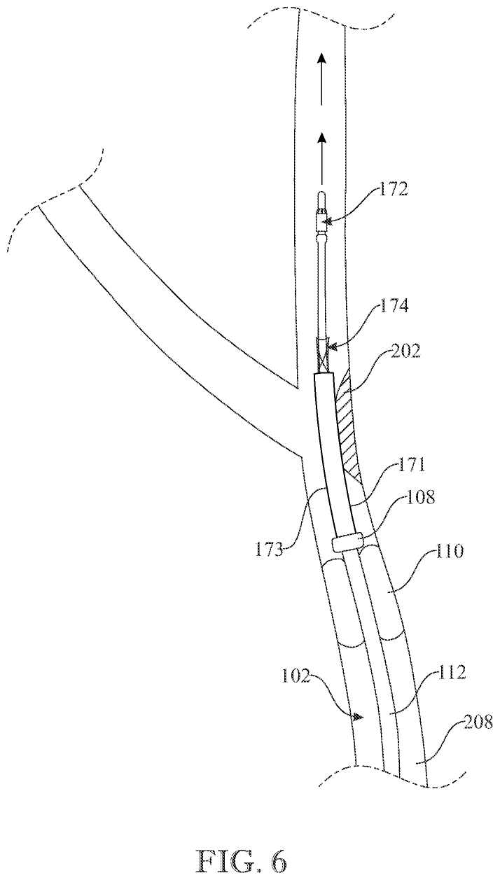

Referring particularly to FIGS. 6 and 7, when the balloon 110 expands to occlude the carotid artery 204, retrograde flow from the common carotid artery to the femoral vein begins to occur. The blood that was once flowing toward the brain through the respective artery begins to flow in the opposite direction and through the femoral sheath 102. The blood flowing through the femoral sheath 102 flows through the entire carotid stenting system 100 and into the venous sheath 146 back into the patient's femoral vein 206. Any impurities, such as plaque, calcium deposits or the like that break off the lesion 202 during the procedure and are flowing through the system are filtered by the filtering device 138, thereby ensuring no impurities, which may cause a stroke or partial blockage, to reenter the blood stream. In one exemplary embodiment, if the retrograde blood flow is not strong enough, a pump system 188 may be used to increase retrograde blood flow. In that particular case, flow reversal may be achieved by connecting the pump 182 and filter system 138 to the venous sheath stopcock with the stopcock valve in the open position. This will establish flow reversal from the carotid artery to the femoral vein. The pump system 188 may be used to control and increase the retrograde blood flow from the carotid artery 204 through the system 100 and into the femoral vein 206. Moreover, the pump 182 of the pump system 188 may be battery operated.

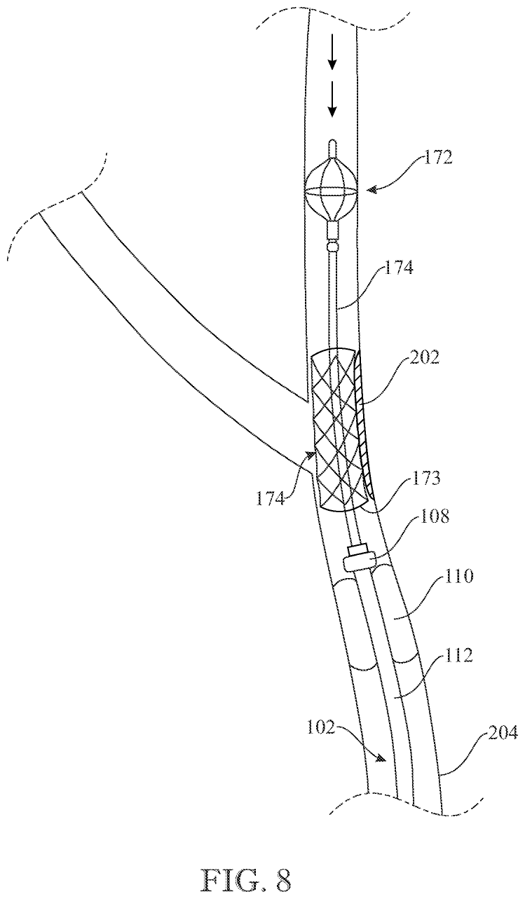

After retrograde blood flow has been established and the filter 172 is deployed, stent 174 is introduced through the valve 119 over the filter and wire (or if no filter present over the wire alone), into the lumen of the femoral sheath 102 and inside of the femoral artery 208. The stent is then advanced until it reaches the lesion 202 into the carotid artery 204. A balloon can be utilized to dilate the lesion before the stent is advanced. The balloon may also be used to “post dilate” the stent to achieve adequate stent-carotid wall opposition. As is shown in FIGS. 7 and 8, after the lesion 202 is compressed against the wall surface of the carotid artery to expand the narrowed artery (i.e., increase blood flow), the stent 174 is deployed at the level of the lesion 202. Once the stent 174 is fully deployed in the carotid artery 204 with a satisfactory result, the balloon 110 occluding the artery 204 is deflated after all equipment, i.e., the filter 172, wire 171, and catheter 173 are removed from the patient. Subsequently, the venous sheath 146 and femoral sheath 102 are also removed from the patient, leaving behind small punctures on the patient's skin that can managed with manual compression or a closure device of the operator's choice, such as a bandage, and covered with additional sterile bandages.

In summary, the carotid stenting system 100 is a new and novel system and method for carotid stenting that is minimally invasive. The method does not require any surgical incisions of the neck of the patient undergoing the procedure and the procedure does not require general anesthesia. The system may utilizes least one filtering devices to protect the person from a stroke or embolism that may be caused by a dislodged particulate matter, such as small particles of calcium or cholesterol plaque, that may occlude the vessel or migrate to the brain from the carotid artery. The venous filter system is designed to prevent embolization to the lungs. One filter device is positioned superior to the lesion to prevent an impurity from entering the brain, and the second filter device may be used to prevent an impurity from reentering the person's blood stream and going to the lungs. The present invention decreases the risk of a stroke. This system serves to improve upon existing systems and methods to treat carotid artery stenosis such as conventional carotid artery stenting and carotid endarterectomy. Current carotid endarterectomy register a stroke rate of about 2-4% within 30 days of the procedure. The present invention is expected to drop the stroke rate to less than 2% within 30 days of the procedure, dramatically decreasing the stroke rate of a patient. Moreover, the procedure will serve to decrease other associated comorbidities, decrease patient discomfort, decrease hospital length stay, and procedure cost.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A carotid stenting system for carotid artery stent placement through trans-femoral access, comprising:
a sheath introducible into a femoral artery at an access site, the sheath providing a general tubular body defining a lumen and having a proximal end, a distal end, a sidewall construction that includes an embedded passageway, and a complaint balloon positioned proximate to the sheath's distal end,
wherein the sheath is of a sufficient length to reach a carotid artery from the access site;
a venous sheath including a connection port at a proximal end, the venous sheath introducible into a femoral vein;
a flexible tube linking the sheath to the venous sheath creating a retrograde blood flow path from the carotid artery to the femoral vein; and
a filtration device intercepting the retrograde blood flow path and configured to capture impurities traversing the retrograde blood flow path,
wherein the sheath allows the advancement of a plurality of devices through the sheath's lumen needed to place a stent in the carotid artery.

2. The carotid stenting system of claim 1, further comprising a connection hub at a proximal end of the sheath, the connection hub including a plurality of connection ports.

3. The carotid stenting system of claim 2, wherein the connection hub includes an inflation port in communication with the passageway of the sheath and connectable to an inflation device storing an injectable fluid that when pushed into the passageway of the sheath, fluid volume inflates the complaint balloon to occlude the carotid artery and prevent antegrade flow in the carotid artery.

4. The carotid stenting system of claim 1, wherein the filtration device includes a pump device that promotes retrograde blood flow through the retrograde blood flow path.

5. The carotid stenting system of claim 1, wherein the sheath is percutaneously cannulated into the femoral artery.

6. The carotid stenting system of claim 1, wherein the sheath is percutaneously cannulated into the femoral artery in retrograde fashion a distance inferior to a patient's inguinal ligament.

7. The carotid stenting system of claim 1, wherein the sheath is percutaneously cannulated into the femoral artery in retrograde fashion about 2 cm to about 4 cm inferior to a patient's inguinal ligament.

8. The carotid stenting system of claim 1, wherein the venous sheath is percutaneously cannulated to the femoral vein.

9. The carotid stenting system of claim 1, wherein the venous sheath is an 8 F sheath percutaneously cannulated to the femoral vein along venous blood flow.

10. The carotid stenting system of claim 1, wherein the sheath includes a marker at the distal end, the marker visible to imaging equipment.

11. The carotid stenting system of claim 1, wherein the sheath is about 90 cm long.

12. A carotid stenting system for carotid artery stent placement through trans-femoral access, comprising:
a sheath percutaneously cannulated into a femoral artery at an access site, the sheath providing a general tubular body defining a lumen and having a proximal end, a distal end, a sidewall construction that includes an embedded passageway, and a complaint balloon positioned proximate to the sheath's distal end,
wherein the sheath is of a sufficient length to reach a carotid artery from the access site;
a venous sheath including a connection port at a proximal end, the venous sheath percutaneously cannulated into a femoral vein;
a flexible tube linking the sheath to the venous sheath creating a retrograde blood flow path from the carotid artery to the femoral vein;
a filtration device intercepting the retrograde blood flow path configured to capture impurities traversing the retrograde blood flow path; and
a stent containment member slidably positioned over a self-expanding stent and introducible into the carotid artery through the lumen of the sheath,
wherein the stent containment member maintains the self-expanding stent in a collapsed state, removal of the stent containment member allows the self-expanding stent to expand in the carotid artery and stent a lesion.

13. The carotid stenting system of claim 12, wherein the sheath includes a connection hub having an inflation port in communication with the passageway of the sheath and connectable to an inflation device storing an injectable fluid that when pushed into the passageway of the arterial sheath fluid volume inflates the complaint balloon to occlude the carotid artery and prevent antegrade flow in the carotid artery.

14. The carotid stenting system of claim 12, wherein the filtration device includes a pump device that promotes retrograde blood flow through the retrograde blood flow path.

15. The carotid stenting system of claim 12, wherein the sheath is percutaneously cannulated into the femoral artery in retrograde fashion a distance inferior to a patient's inguinal ligament.

16. The carotid stenting system of claim 12, wherein the arterial sheath is about 90 cm long.

17. A carotid stenting system for carotid artery stent placement through trans-femoral access, comprising:
a removable arterial sheath percutaneously cannulated into a femoral artery at an access site;
a venous sheath including a connection port at a proximal end, the venous sheath percutaneously cannulated into a femoral vein along the venous blood flow;
a first removable stiff wire introducible through the removable arterial sheath into the femoral artery, the first stiff wire of a sufficient length to reach the carotid artery from the access site,
wherein the removable arterial sheath is removed after the first stiff wire is introduced into the carotid artery;
a removable catheter slidable over the wire and insertable into the femoral artery through the removable arterial sheath and of a sufficient length to reach the carotid artery from the access site;
a second removable stiff wire introducible through the removable catheter into the femoral artery, the second wire of a sufficient length to reach the carotid artery from the access site,
wherein the second stiff wire is introduced into the removable catheter after the first stiff wire is removed, and wherein the removable catheter is removed after the second stiff wire is introduced into the carotid artery;
a sheath percutaneously cannulated into the femoral artery at the access and slidable over the second stiff wire, the sheath providing a general tubular body defining a lumen and having a proximal end, a distal end, a sidewall construction that includes an embedded passageway, and a complaint balloon positioned proximate to the sheath's distal end,
wherein the sheath is of a sufficient length to reach a carotid artery from the access site, and
wherein the second stiff wire is removed after the sheath is introduced into the carotid artery;
a visible marker at the distal end of the sheath that is visible to imaging equipment;
a connection hub at the proximal end of the sheath, the connection hub including a plurality of connection ports;
a flexible tube linking the sheath to the venous sheath creating a retrograde blood flow path from the carotid artery to the femoral vein;
at least one control valve connected to the flexible tubing to regulate retrograde blood flow through the retrograde blood flow path from the carotid artery to the femoral vein;
a filtration device intercepting the retrograde blood flow path configured to capture impurities traversing the retrograde blood flow path; and
a pump device connected to the filtration device to promote retrograde blood flow from the carotid artery through the arterial sheath, through the flexible tubing, pass the filtration device, through the venous sheath and into the femoral vein; and
a stent containment member slidably positioned over a self-expanding stent and introducible into the carotid artery through the lumen of the sheath,
wherein the stent containment member maintains the self-expanding stent in a collapsed state, removal of the stent containment member allows the self-expanding stent to expand in the carotid artery and stent a lesion.

18. The carotid stenting system of claim 17, wherein the connection hub includes an inflation port in communication with the passageway of the sheath and connectable to an inflation device storing an injectable fluid that when pushed into the passageway of the sheath, fluid volume inflates the complaint balloon to occlude the carotid artery and prevent antegrade flow in the carotid artery.

19. The carotid stenting system of claim 17, wherein the arterial sheath is about 90 cm long.

20. The carotid stenting system of claim 17, wherein the removable arterial sheath is percutaneously cannulated into the femoral artery in retrograde fashion a distance inferior to a patient's inguinal ligament, and wherein the arterial sheath is about 12 cm 6 F sheath and the venous sheath is an 8 F sheath.
